(19) United States
(12) Patent Application Publication
(10) Pub. No.: US 2026/0237478 A1
(43) Pub. Date: Aug. 13, 2026

(54) ARTIFICIAL INTELLIGENCE-BASED GENERATOR FOR SYNTHETIC MEDICAL CHARTS

(71) Applicant: UpToDate, Inc., Waltham, MA (US)

(72) Inventors: Andrew Mulder, Ottawa (CA); Adam Rabinowitz, Waltham, MA (US)

(73) Assignee: UpToDate, Inc., Waltham, MA (US)

(21) Appl. No.: 19/274,993

(22) Filed: Jul. 21, 2025

Related U.S. Application Data
(60) Provisional application No. 63/757,628, filed on Feb. 12, 2025.

(51) Int. Cl. G16H 15/00; G06F 8/35; G16H 10/60; H04L 9/40

(52) U.S. Cl. CPC G16H 15/00; G06F 8/35; G16H 10/60; H04L 63/0428

(57) ABSTRACT

Creating a requested number of medical record forms, each representing a fictitious encounter between one fictitious patent and a provider, includes redacting an authentic medical chart, identifying parameters, to be filled in, from the authentic or redacted medical chart, and converting the redacted authentic medical chart to the template. An LLM is prompted, with information relating to the first user parameter and a medical charting instruction, to generate a first unit of content relating to the first parameter. A chart memory includes a summary, generated by the LLM, of the first written content, and a number of units of written content created. If the number of units of written content created is less than the requested number, prompting the LLM to generate a subsequent unit of written content, using the summary to ensure that the subsequent unit is temporally, logically and medically consistent with the first unit of written content.

### RELATED AND PRIORITY APPLICATION

This application claims priority to U.S. Provisional Application No. 63/757,628, filed Feb. 12, 2025, which is hereby incorporated by reference in its entirety.

### TECHNICAL FIELD

This application relates generally to document generation via artificial intelligence, and more particularly, to generating properly formatted synthetic medical charts with authentically appearing content.

### BACKGROUND

Software applications that analyze or otherwise interact with medical chart documents require testing. Before such applications can be released to be used on authentic and current medical charts, a corpus of medical chart documents must be used to test the application. However, real medical charts contain protected health information (“PHI”) and/or personally identifiable information (“PII”) which are covered by substantial regulation for storage and use.

Therefore, it would be helpful to use fictitious medical charts for testing. However, creating fictitious medical charts can be a time-consuming process, and/or the existing universe of manually created fictitious medical charts can be too limited for testing purposes.

Accordingly, it would be advantageous to be able to automatically create fictitious medical charts, which contain information specific enough to allow for substantive health care applications, that analyze the content of the chart, to be tested. It would also be advantageous to be able to create fictitious medical charts that are medically consistent, from chart to chart, for the same fictitious patient over fictitious time.

### SUMMARY

A computer-implemented method for creating a requested number of medical record forms relating to a single fictitious patient, each of the medical record forms representing a fictitious encounter between the fictitious patent and a medical provider, is herein disclosed. The method includes creating a medical chart template, the creating a medical chart template further comprising: redacting personally identifiable information from an authentic medical chart to create a redacted authentic medical chart; identifying a first user parameter and a second user parameter, to be filled in, from the authentic medical chart or from the redacted authentic medical chart; and converting an image of the redacted authentic medical chart to HTML code representing the medical chart template.

The method further includes generating synthetic medical content, the generating the synthetic medical content further includes prompting a large language model (“LLM”) to generate a first unit of written content for one of the requested number of medical forms, the prompt containing information relating to the first user parameter and an instruction relating to medical charting practice, the written content containing content relating to the first user parameter; creating a chart memory for the fictitious patient, the chart memory including a summary, generated by the LLM, of the first written content, and a number of units of written content created; upon a determination that the number of units of written content created is less than the requested number of medical forms, prompting the LLM to generate a subsequent unit of written content, the prompt containing the information relating to a second user parameter, an instruction relating to medical charting practice, and the summary from the chart memory; and updating the summary to contain a summary of information from the subsequent unit of written content.

The method may also include the step of creating a first synthetic medical chart in the form of the medical chart template, containing the first unit of written content in a location, within the synthetic medical chart, associated with the first user parameter. The method may also include the step of creating a synthetic medical chart further comprises prompting the LLM to generate the synthetic medical chart, the prompt containing the first user parameter, the medical chart template, and the first unit of written content.

The method may also include the step of, upon a determination of the existence of the subsequent unit of written content, and upon a determination that a synthetic medical chart containing the subsequent unit of written content has not been created, creating a second synthetic medical chart in the form of the medical chart template, containing the subsequent unit of written content in a location within the synthetic medical chart associated with the second user parameter.

The method may also include the step of creating a first synthetic medical chart further comprises prompting the LLM to create first HTML code in accordance with the medical chart template and containing the first unit of written content. The step of creating a second synthetic medical chart may also include prompting the LLM to create second HTML code in accordance with the medical chart template and containing the second unit of written content. The method may also include creating a PDF containing a conversion of the output of the first HTML code and the second HTML code to PDF format.

### BRIEF DESCRIPTION OF THE DRAWINGS

The features and advantages of the present invention will be more fully disclosed in, or rendered obvious by the following detailed description of the preferred embodiments, which are to be considered together with the accompanying drawings wherein like numbers refer to like parts and further wherein:

FIG. 1 illustrates a network environment configured to provide synthetic medical chart creation, in accordance with some embodiments;

FIG. 2 illustrates a computer system configured to implement one or more processes, in accordance with some embodiments;

FIG. 3 is a visual depiction of the creation of a synthetic medical chart from a medical chart template, in accordance with some embodiments.

FIG. 4A is a process flow illustrating various steps of a process for converting a PDF redacted medical record to an HTML template, in accordance with some embodiments.

FIG. 4B is a process flow illustrating various steps of a process for generating content for synthetic medical charts, in accordance with some embodiments.

FIG. 4C is a process flow illustrating various steps of a process for merging an HTML template with synthetic generated content, and generating a PDF of a synthetic medical chart, in accordance with some embodiments.

FIG. 5A illustrates an artificial neural network, in accordance with some embodiments;

FIG. 5B illustrates a tree-based artificial neural network, in accordance with some embodiments;

FIG. 5C illustrates a deep neural network (DNN), in accordance with some embodiments;

FIG. 6A is a flowchart illustrating a training method for generating a trained machine learning model, in accordance with some embodiments; and

FIG. 6B is a process flow illustrating various steps of the training method of FIG. 6A, in accordance with some embodiments.

FIGS. 7A and 7B are a flow diagram of a method in accordance with some embodiments.

### DETAILED DESCRIPTION

This description of the exemplary embodiments is intended to be read in connection with the accompanying drawings, which are to be considered part of the entire written description. Terms concerning data connections, coupling and the like, such as “connected” and “interconnected,” and/or “in signal communication with” refer to a relationship wherein systems or elements are electrically connected (e.g., wired, wireless, etc.) to one another either directly or indirectly through intervening systems, unless expressly described otherwise. The term “operatively coupled” is such a coupling or connection that allows the pertinent structures to operate as intended by virtue of that relationship.

In the following, various embodiments are described with respect to the claimed systems as well as with respect to the claimed methods. Features, advantages, or alternative embodiments herein may be assigned to the other claimed objects and vice versa. In other words, claims for the systems may be improved with features described or claimed in the context of the methods. In this case, the functional features of the method are embodied by objective units of the systems. While the present disclosure is susceptible to various modifications and alternative forms, specific embodiments are shown by way of example in the drawings and will be described in detail herein. The objectives and advantages of the claimed subject matter will become more apparent from the following detailed description of these exemplary embodiments in connection with the accompanying drawings.

Furthermore, in the following, various embodiments are described with respect to a specialized system to generate synthetic medical charts by a combination of calling a Large Language Model (LLM) and prompting it to execute targeted tasks and carrying out functions through the coding language. In various embodiments, the system may execute tasks including, but not limited to converting PDF pages to PNG images, calling the LLM to execute tasks, calling an image model to generate logo files, updating chart memory for the first patient, updating chart memory summary for the succeeding patients, processing and enhancing HTML charts and converting them to PDFs.

A real electronic medical chart is redacted by the user which may refer to the removing of the patient's personal health information from the medical chart due to privacy concerns or compliance with regulations. The system may then convert the PDF pages of the real electronic medical chart to PNG images. The PDF pages may contain various layouts such as tables, images, and formatting which may make it hard for the LLM to interpret the text. Converting to PNG images makes it easier for the LLM to interpret and analyze due to clear visual representation of the text.

The system may then call the LLM to generate a prompt to convert the PNG images to HTML code by providing it with necessary information and guidelines. The PNG images are then fed to the LLM. The LLM converts the PNG images to generate corresponding HTML code with the same layout and format. The LLM writes the HTML code for the medical chart template by analyzing the data and structure of the retracted medical chart initially fed to it. The HTML code may have the same font size, font style, CSS styles, alignment, image placeholder, background color as the document. The HTML code forms a HTML webpage which is a template of the retracted real medical chart.

The system may then send the HTML template, report creation instructions, user parameters including but not limited to patient name, facility name, facility address, facility phone, date of birth, birth sex, number of encounters, current gender, service date, and visit type to the LLM. The LLM may then be given a prompt to create a fictious textual, e.g. plain text, content for the HTML template. The HTML template is provided to the LLM for reference on what content it should generate. The instructions on how to create the best report may refer to detailed guidelines on what a medical chart should contain. The user parameters may or may not be provided by the system. The LLM might generate the user parameters if they are not provided by the system. Summaries of past visits may be provided to logically create fictional content and if not, the system might consider it as the first visit.

The LLM processes and assimilates all the relevant information and data. The LLM generates the first encounter. The system may create a chart memory to store user parameters from the first encounter. The chart memory may then be fed to the LLM during each subsequent encounter. The chart memory facilitates the logical generation of encounters by providing the LLM with important patient details on each encounter. The system may create a chart memory summary for managing multiple encounters. The system may then prompt the LLM to create summaries of each encounter after they are generated. The summaries might be stored in the chart memory summary and inputted to the LLM during each encounter to support logical and consistent generation of content. In some embodiments, the encounters are generated in plain text and contain fictional content generated systematically for the synthetic medical charts. Markdown is a text format that may be used in the invention, for seamless combination with an HTML webpage by adding the text inside the HTML tags of the webpage. Other text formats may be used instead of or in addition to markdown.

The system provides and prompts the LLM to integrate the HTML template, fictional content, user parameters including, but not limited to number of encounters, patient name, facility name, facility address, facility phone, date of birth, birth sex, current gender, service date and visit type. The LLM fills in the HTML template with the fictional content while adhering to the instructions and using the user parameters if provided and generating them if not. The HTML chart is generated with synthetic content for each encounter.

The system processes the HTML chart to have consistent CSS styling by applying the same CSS styling as a real medical chart. The system may add a logo image to each HTML chart. The logo image may be generated using image model to create image from text using deep learning methodologies. The system may convert the HTML chart to a single PDF chart. The system may do this step to depict real medical charts which are designed to be one single PDF with multiple encounters. The PDF chart may be enhanced with, but not limited to, image noise and other artifacts. Image noise might be added to the PDF chart to make it similar to real medical charts. Once the system has finished executing the process, the final output—a synthetic medical chart—can be produced in PDF format with the required number of encounters.

In some embodiments, systems, and methods for generating synthetic medical charts includes one or more trained LLMs.

In general, a trained function mimics cognitive functions that humans associate with other human minds. In particular, by training based on training data the trained function is able to adapt to new circumstances and to detect and extrapolate patterns.

In general, parameters of a trained function may be adapted by means of training. In particular, a combination of supervised training, semi-supervised training, unsupervised training, reinforcement learning and/or active learning may be used. Furthermore, representation learning (an alternative term is “feature learning”) may be used. In particular, the parameters of the trained functions may be adapted iteratively by several steps of training.

In some embodiments, a trained function may include a neural network, a support vector machine, a decision tree, a Bayesian network, a clustering network, Qlearning, genetic algorithms and/or association rules, and/or any other suitable artificial intelligence architecture. In some embodiments, a neural network may be a deep neural network, a convolutional neural network, a convolutional deep neural network, etc. Furthermore, a neural network may be an adversarial network, a deep adversarial network, a generative adversarial network, etc.

In various embodiments, neural networks which are trained (e.g., configured or adapted) to generate synthetic medical charts, are disclosed. A trained LLM may be configured to receive a set of input data, such as prompts, templates, summaries, and the like.

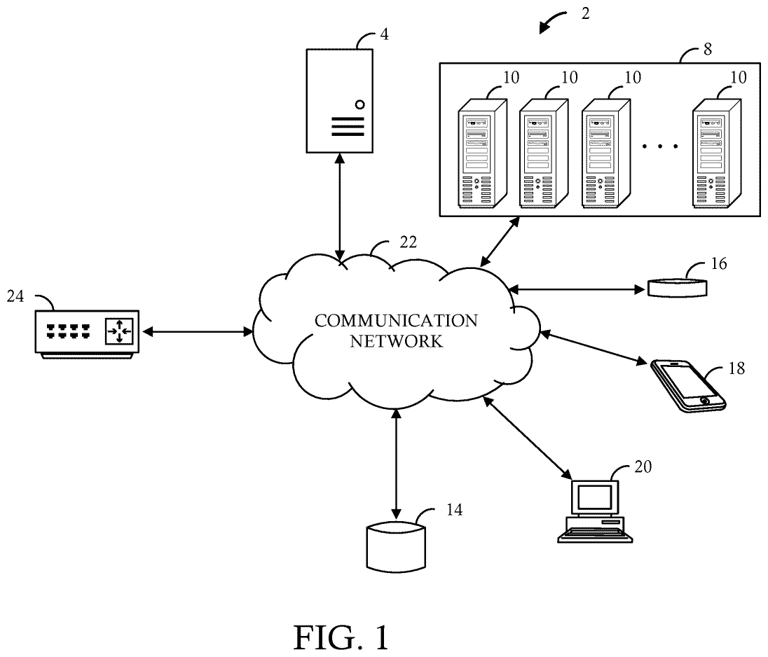

FIG. 1 illustrates a network environment 2 configured to provide a system for the creation of synthetic medical charts, in accordance with some embodiments. The network environment 2 includes a plurality of devices or systems configured to communicate over one or more network channels, illustrated as a network cloud 22. For example, in various embodiments, the network environment 2 may include, but is not limited to, a management server 4, a cloud-based engine 8 including one or more processing devices 10, a database 14, and/or one or more user computing devices 16, 18, 20 operatively coupled over the network 22. The management server 4, the processing device(s) 10, and/or the user computing devices 16, 18, 20 may each be a suitable computing device that includes any hardware or hardware and software combination for processing and handling information. For example, each computing device may include, but is not limited to, one or more processors, one or more field-programmable gate arrays (FPGAs), one or more application-specific integrated circuits (ASICs), one or more state machines, digital circuitry, and/or any other suitable circuitry. In addition, each computing device may transmit and receive data over the communication network 22.

In some embodiments, each of the management server 4 and the processing device(s) 10 may be a computer, a workstation, a laptop, a server such as a cloud-based server, or any other suitable device. In some embodiments, each of the processing devices 10 is a server that includes one or more processing units, such as one or more graphical processing units (GPUs), one or more central processing units (CPUs), and/or one or more processing cores. Each processing device 10 may, in some embodiments, execute one or more virtual machines. In some embodiments, processing resources (e.g., capabilities) of the one or more processing devices 10 are offered as a cloud-based service (e.g., cloud computing). For example, the cloud-based engine 8 may offer computing and storage resources of the one or more processing devices 10 to the management server 4.

In some embodiments, each of the user computing devices 16, 18, 20 may be a cellular phone, a smart phone, a tablet, a personal assistant device, a voice assistant device, a digital assistant, a laptop, a computer, or any other suitable device. In some embodiments, the management server 4, and/or the processing devices 10 are operated by the network environment provider, and the user computing devices 16, 18, 20 are operated by users of the network environment. In some embodiments, the processing devices 10 are operated by a third party (e.g., a cloud-computing provider).

Although FIG. 1 illustrates three user computing devices 16, 18, 20, the network environment 2 may include any number of user computing devices 16, 18, 20. Similarly, the network environment 2 may include any number of the management server 4, the processing devices 10, and/or the databases 14. It will further be appreciated that additional systems, servers, storage mechanism, etc. may be included within the network environment 2. In addition, although embodiments are illustrated herein having individual, discrete systems, it will be appreciated that, in some embodiments, one or more systems may be combined into a single logical and/or physical system. For example, in various embodiments, one or more of the management server 4, the database 14, the user computing devices 16, 18, 20, and/or the router 24 may be combined into a single logical and/or physical system. Similarly, although embodiments are illustrated having a single instance of each device or system, it will be appreciated that additional instances of a device may be implemented within the network environment 2. In some embodiments, two or more systems may be operated on shared hardware in which each system operates as a separate, discrete system utilizing the shared hardware, for example, according to one or more virtualization schemes.

The communication network 22 may be a WiFi® network, a cellular network such as a 3GPP® network, a Bluetooth® network, a satellite network, a wireless local area network (LAN), a network utilizing radio-frequency (RF) communication protocols, a Near Field Communication (NFC) network, a wireless Metropolitan Area Network (MAN) connecting multiple wireless LANs, a wide area network (WAN), or any other suitable network. The communication network 22 may provide access to, for example, the Internet.

The management server 4 is further operable to communicate with the database 14 over the communication network 22. For example, the management server 4 may store data to, and read data from, the database 14. The database 14 may be a remote storage device, such as a cloud-based server, a disk (e.g., a hard disk), a memory device on another application server, a networked computer, or any other suitable remote storage. Although shown remote to the management server 4, in some embodiments, the database 14 may be a local storage device, such as a hard drive, a non-volatile memory, or a USB stick.

In some embodiments, the management server 4 generates training data for a plurality of models (e.g., machine learning models, deep learning models, statistical models, algorithms, etc.) based on aggregation data, variant-level data, holiday and event data, recall data, historical user session data, search data, purchase data, catalog data, advertisement data for the users, etc. The management server 4 and/or one or more of the processing devices 10 may train one or more models based on corresponding training data. The management server 4 may store the models in a database, such as in the database 14 (e.g., a cloud storage database).

The models, when executed by the management server 4, allow the management server 4 to process and generate synthetic medical charts. For example, the management server 4 may obtain one or more models from the database 14. The management server 4 may then receive, in real-time from the user computing devices 16, 18, 20, a prompt, which may include a template, one or more user parameters, instructions relating to medical charting practice, and the like, as described in further detail below. In response to receiving prompt, the management server 4 may execute one or more models to generate a synthetic medical chart, using the methods described below.

In some embodiments, the management server 4 assigns the models (or parts thereof) for execution to one or more processing devices 10. For example, each model may be assigned to a virtual machine hosted by a processing device 10. The virtual machine may cause the models or parts thereof to execute on one or more processing units such as GPUs. In some embodiments, the virtual machines assign each model (or part thereof) among a plurality of processing units. Based on the output of the models, management server 4 may generate synthetic medical charts, which may then be stored for testing purposes, e.g. in database 14. In some embodiments, the models may be run by cloud based engine 8 including one or more processing devices. In some embodiments, management server may then call the models by sending, e.g. the prompt, to cloud based engine 8 via communication network 22.

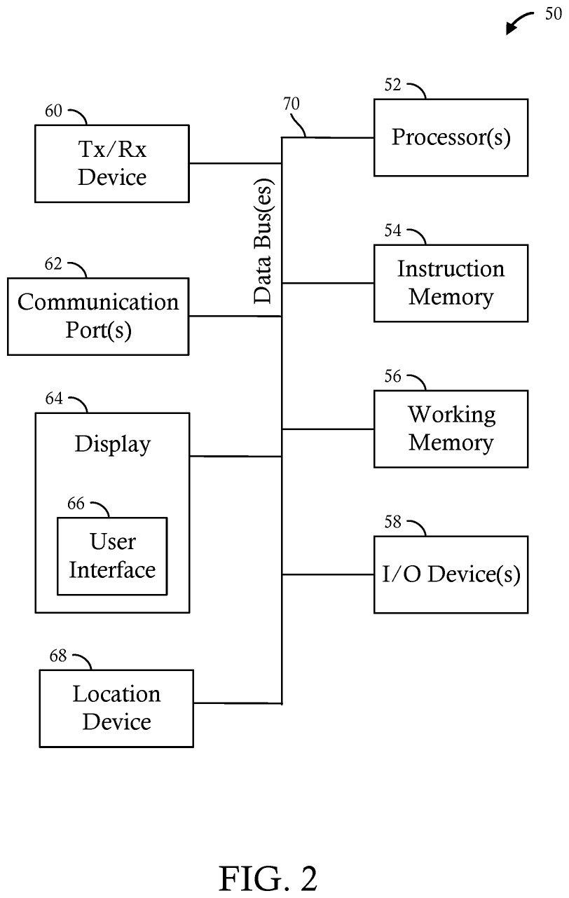

FIG. 2 illustrates a block diagram of a computing device 50, in accordance with some embodiments. In some embodiments, each of the management server 4, the one or more processing devices 10, the workstation(s) 12, and/or the user computing devices 16, 18, 20 in FIG. 1 may include the features shown in FIG. 2. Although FIG. 2 is described with respect to certain components shown therein, it will be appreciated that the elements of the computing device 50 may be combined, omitted, and/or replicated. In addition, it will be appreciated that additional elements other than those illustrated in FIG. 2 may be added to the computing device.

As shown in FIG. 2, the computing device 50 may include one or more processors 52, an instruction memory 54, a working memory 56, one or more input/output devices 58, a transceiver 60, one or more communication ports 62, a display 64 with a user interface 66, and an optional location device 68, all operatively coupled to one or more data buses 70. The data buses 70 allow for communication among the various components. The data buses 70 may include wired, or wireless, communication channels.

The one or more processors 52 may include any processing circuitry operable to control operations of the computing device 50. In some embodiments, the one or more processors 52 include one or more distinct processors, each having one or more cores (e.g., processing circuits). Each of the distinct processors may have the same or different structure. The one or more processors 52 may include one or more central processing units (CPUs), one or more graphics processing units (GPUs), application specific integrated circuits (ASICs), digital signal processors (DSPs), a chip multiprocessor (CMP), a network processor, an input/output (I/O) processor, a media access control (MAC) processor, a radio baseband processor, a co-processor, a microprocessor such as a complex instruction set computer (CISC) microprocessor, a reduced instruction set computing (RISC) microprocessor, and/or a very long instruction word (VLIW) microprocessor, or other processing device. The one or more processors 52 may also be implemented by a controller, a microcontroller, an application specific integrated circuit (ASIC), a field programmable gate array (FPGA), a programmable logic device (PLD), etc.

In some embodiments, the one or more processors 52 are configured to implement an operating system (OS) and/or various applications. Examples of an OS include, for example, operating systems generally known under various trade names such as Apple macOS™, Microsoft Windows™, Android™, Linux™, and/or any other proprietary or open-source OS. Examples of applications include, for example, network applications, local applications, data input/output applications, user interaction applications, etc.

The instruction memory 54 may store instructions that are accessed (e.g., read) and executed by at least one of the one or more processors 52. For example, the instruction memory 54 may be a non-transitory, computer-readable storage medium such as a read-only memory (ROM), an electrically erasable programmable read-only memory (EEPROM), flash memory (e.g. NOR and/or NAND flash memory), content addressable memory (CAM), polymer memory (e.g., ferroelectric polymer memory), phase-change memory (e.g., ovonic memory), ferroelectric memory, silicon-oxide-nitride-oxide-silicon (SONOS) memory, a removable disk, CD-ROM, any non-volatile memory, or any other suitable memory. The one or more processors 52 may be configured to perform a certain function or operation by executing code, stored on the instruction memory 54, embodying the function or operation. For example, the one or more processors 52 may be configured to execute code stored in the instruction memory 54 to perform one or more of any function, method, or operation disclosed herein.

Additionally, the one or more processors 52 may store data to, and read data from, the working memory 56. For example, the one or more processors 52 may store a working set of instructions to the working memory 56, such as instructions loaded from the instruction memory 54. The one or more processors 52 may also use the working memory 56 to store dynamic data created during one or more operations. The working memory 56 may include, for example, random access memory (RAM) such as a static random access memory (SRAM) or dynamic random access memory (DRAM), Double-Data-Rate DRAM (DDR-RAM), synchronous DRAM (SDRAM), an EEPROM, flash memory (e.g. NOR and/or NAND flash memory), content addressable memory (CAM), polymer memory (e.g., ferroelectric polymer memory), phase-change memory (e.g., ovonic memory), ferroelectric memory, silicon-oxide-nitride-oxide-silicon (SONOS) memory, a removable disk, CD-ROM, any non-volatile memory, or any other suitable memory. Although embodiments are illustrated herein including separate instruction memory 54 and working memory 56, it will be appreciated that the computing device 50 may include a single memory unit configured to operate as both instruction memory and working memory. Further, although embodiments are discussed herein including non-volatile memory, it will be appreciated that computing device 50 may include volatile memory components in addition to at least one non-volatile memory component.

In some embodiments, the instruction memory 54 and/or the working memory 56 includes an instruction set, in the form of a file for executing various methods, such as methods for generating synthetic medical charts, as described herein. The instruction set may be stored in any acceptable form of machine-readable instructions, including source code or various appropriate programming languages. Some examples of programming languages that may be used to store the instruction set include, but are not limited to: Java, JavaScript, C, C++, C#, Python, Objective-C, Visual Basic, .NET, HTML, CSS, SQL, NoSQL, Rust, Perl, etc. In some embodiments a compiler or interpreter is configured to convert the instruction set into machine executable code for execution by the one or more processors 52.

The input-output devices 58 may include any suitable device that allows for data input or output. For example, the input-output devices 58 may include one or more of a keyboard, a touchpad, a mouse, a stylus, a touchscreen, a physical button, a speaker, a microphone, a keypad, a click wheel, a motion sensor, a camera, and/or any other suitable input or output device.

The transceiver 60 and/or the communication port(s) 62 allow for communication with a network, such as the communication network 22 of FIG. 1. For example, if the communication network 22 of FIG. 1 is a cellular network, the transceiver 60 is configured to allow communications with the cellular network. In some embodiments, the transceiver 60 is selected based on the type of the communication network 22 the computing device 50 will be operating in. The one or more processors 52 are operable to receive data from, or send data to, a network, such as the communication network 22 of FIG. 1, via the transceiver 60.

The communication port(s) 62 may include any suitable hardware, software, and/or combination of hardware and software that is capable of coupling the computing device 50 to one or more networks and/or additional devices. The communication port(s) 62 may be arranged to operate with any suitable technique for controlling information signals using a desired set of communications protocols, services, or operating procedures. The communication port(s) 62 may include the appropriate physical connectors to connect with a corresponding communications medium, whether wired or wireless, for example, a serial port such as a universal asynchronous receiver/transmitter (UART) connection, a Universal Serial Bus (USB) connection, or any other suitable communication port or connection. In some embodiments, the communication port(s) 62 allows for the programming of executable instructions in the instruction memory 54. In some embodiments, the communication port(s) 62 allow for the transfer (e.g., uploading or downloading) of data, such as machine learning model training data.

In some embodiments, the communication port(s) 62 are configured to couple the computing device 50 to a network. The network may include local area networks (LAN) as well as wide area networks (WAN) including without limitation Internet, wired channels, wireless channels, communication devices including telephones, computers, wire, radio, optical and/or other electromagnetic channels, and combinations thereof, including other devices and/or components capable of/associated with communicating data. For example, the communication environments may include in-body communications, various devices, and various modes of communications such as wireless communications, wired communications, and combinations of the same.

In some embodiments, the transceiver 60 and/or the communication port(s) 62 are configured to utilize one or more communication protocols. Examples of wired protocols may include, but are not limited to, Universal Serial Bus (USB) communication, RS-232, RS-422, RS-423, RS-485 serial protocols, Fire Wire, Ethernet, Fibre Channel, MIDI, ATA, Serial ATA, PCI Express, T-1 (and variants), Industry Standard Architecture (ISA) parallel communication, Small Computer System Interface (SCSI) communication, or Peripheral Component Interconnect (PCI) communication, etc. Examples of wireless protocols may include, but are not limited to, the Institute of Electrical and Electronics Engineers (IEEE) 802.xx series of protocols, such as IEEE 802.11a/b/g/n/ac/ag/ax/be, IEEE 802.16, IEEE 802.20, GSM cellular radiotelephone system protocols with GPRS, CDMA cellular radiotelephone communication systems with 1×RTT, EDGE systems, EV-DO systems, EV-DV systems, HSDPA systems, Wi-Fi Legacy, Wi-Fi 1/2/3/4/5/6/6E, wireless personal area network (PAN) protocols, Bluetooth Specification versions 5.0, 6, 7, legacy Bluetooth protocols, passive or active radio-frequency identification (RFID) protocols, Ultra-Wide Band (UWB), Digital Office (DO), Digital Home, Trusted Platform Module (TPM), ZigBee, etc.

The display 64 may be any suitable display, and may display the user interface 66. The user interfaces 66 may enable user interaction with the system for creating and/or using synthetic medical charts. For example, the user interface 66 may be a user interface for an application for testing other software applications that are designed to evaluate or otherwise interact with authentic medical charts. In some embodiments, a user may interact with the user interface 66 by engaging the input-output devices 58. In some embodiments, the display 64 may be a touchscreen, where the user interface 66 is displayed on the touchscreen.

The display 64 may include a screen such as, for example, a Liquid Crystal Display (LCD) screen, a light-emitting diode (LED) screen, an organic LED (OLED) screen, a movable display, a projection, etc. In some embodiments, the display 64 may include a coder/decoder, also known as Codecs, to convert digital media data into analog signals. For example, the visual peripheral output device may include video Codecs, audio Codecs, or any other suitable type of Codec.

The optional location device 68 may be communicatively coupled to a location network and operable to receive position data from the location network. For example, in some embodiments, the location device 68 includes a GPS device configured to receive position data identifying a latitude and longitude from one or more satellites of a GPS constellation. As another example, in some embodiments, the location device 68 is a cellular device configured to receive location data from one or more localized cellular towers. Based on the position data, the computing device 50 may determine a local geographical area (e.g., town, city, state, etc.) of its position.

In some embodiments, the computing device 50 is configured to implement one or more modules or engines, each of which is constructed, programmed, configured, or otherwise adapted, to autonomously carry out a function or set of functions. A module/engine may include a component or arrangement of components implemented using hardware, such as by an application specific integrated circuit (ASIC) or field-programmable gate array (FPGA), for example, or as a combination of hardware and software, such as by a microprocessor system and a set of program instructions that adapt the module/engine to implement the particular functionality, which (while being executed) transform the microprocessor system into a special-purpose device. A module/engine may also be implemented as a combination of the two, with certain functions facilitated by hardware alone, and other functions facilitated by a combination of hardware and software. In certain implementations, at least a portion, and in some cases, all, of a module/engine may be executed on the processor(s) of one or more computing platforms that are made up of hardware (e.g., one or more processors, data storage devices such as memory or drive storage, input/output facilities such as network interface devices, video devices, keyboard, mouse or touchscreen devices, etc.) that execute an operating system, system programs, and application programs, while also implementing the engine using multitasking, multithreading, distributed (e.g., cluster, peer-peer, cloud, etc.) processing where appropriate, or other such techniques. Accordingly, each module/engine may be realized in a variety of physically realizable configurations, and should generally not be limited to any particular implementation exemplified herein, unless such limitations are expressly called out. In addition, a module/engine may itself be composed of more than one sub-modules or sub-engines, each of which may be regarded as a module/engine in its own right. Moreover, in the embodiments described herein, each of the various modules/engines corresponds to a defined autonomous functionality; however, it should be understood that in other contemplated embodiments, each functionality may be distributed to more than one module/engine. Likewise, in other contemplated embodiments, multiple defined functionalities may be implemented by a single module/engine that performs those multiple functions, possibly alongside other functions, or distributed differently among a set of modules/engines than specifically illustrated in the embodiments herein.

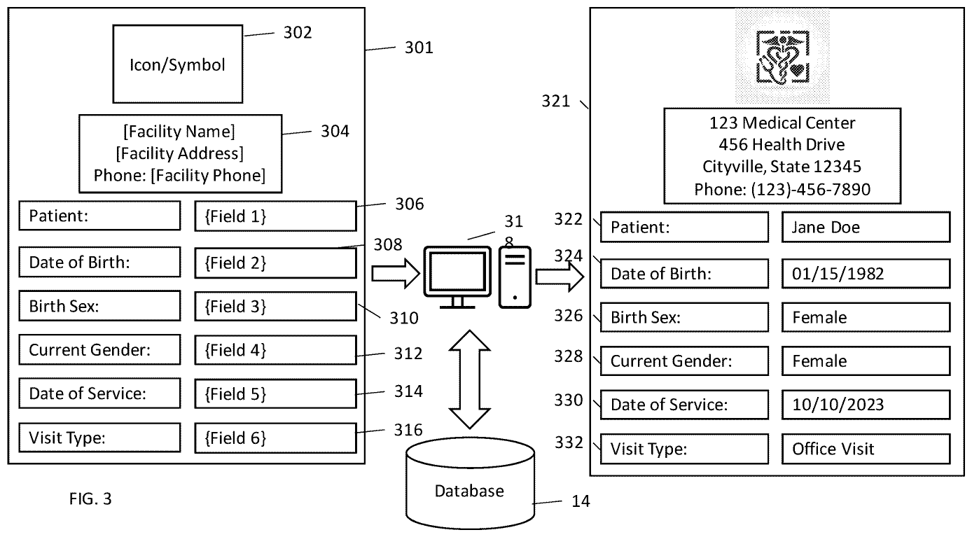

Turning now to FIG. 3 is a visual depiction of converting a template of an initial information page of a medical chart to an initial information page with synthetic content, generally designated as 300. The initial information page of a medical chart is generally designated 301 and also referred to herein as the HTML face sheet. The process may include attaining the redacted HTML face sheet 301.

The redacted HTML sheet 301 may include an icon/symbol placeholder 302 that may allow the system to add a logo image to enhance the HTML sheet. The logo image may be generated by an image generative artificial intelligence model by converting text to image using deep machine learning procedure. The logo image may also be obtained from the template, from which it may be extracted and then later re-inserted as discussed below.

The redacted HTML sheet 301 may comprise a facility information block 304 that may contain the facility name, facility address and the facility phone number. The facility information may be provided by database 14 to the programming software/platform on the computing device 16, 18, or 20 of FIG. 1. The facility information may then be added to the redacted HTML face sheet 201.

The redacted HTML sheet 301 may consist of placeholders for the user parameters that may be for the patient 306, date of birth 308, birth sex 310, current gender 312, date of service 314 and visit type 316. All of the user parameters may be stored in the database 14 and provided to the programming software/platform to be integrated with the HTML sheet to generate the final synthetic medical record 321, containing synthetic medical chart content.

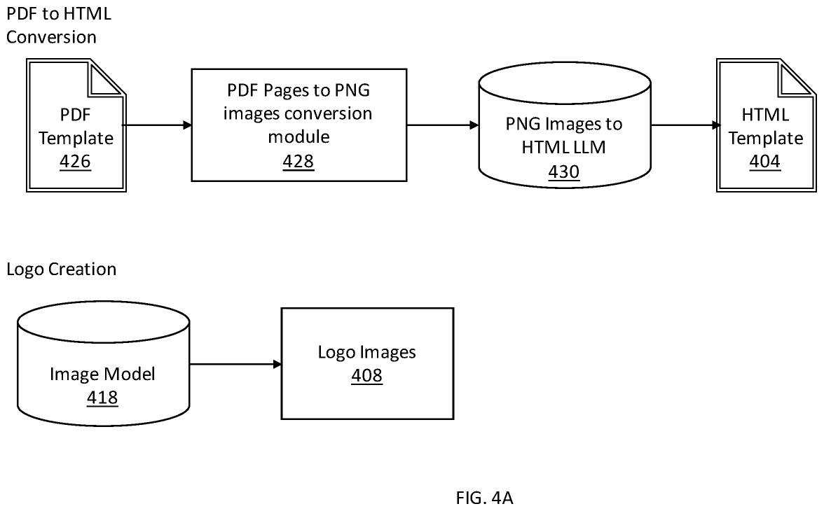

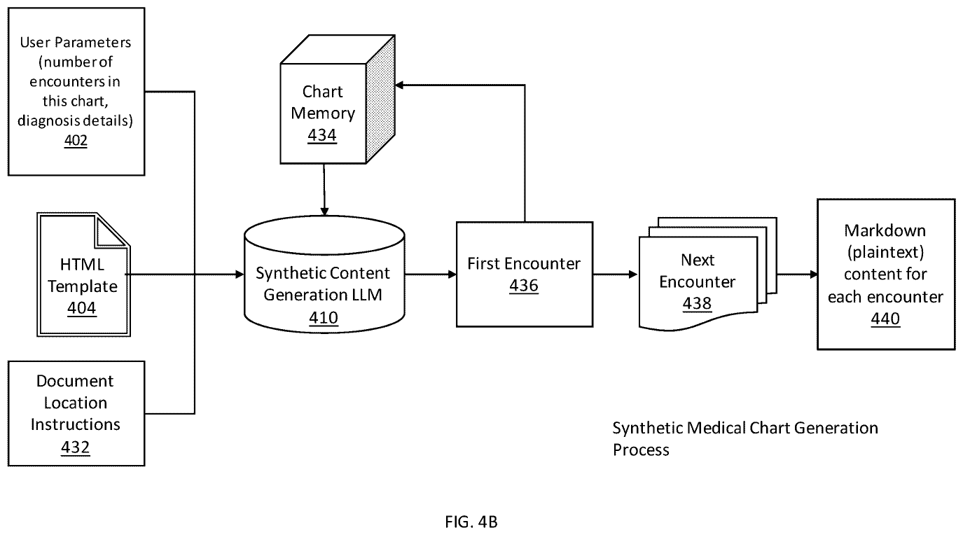

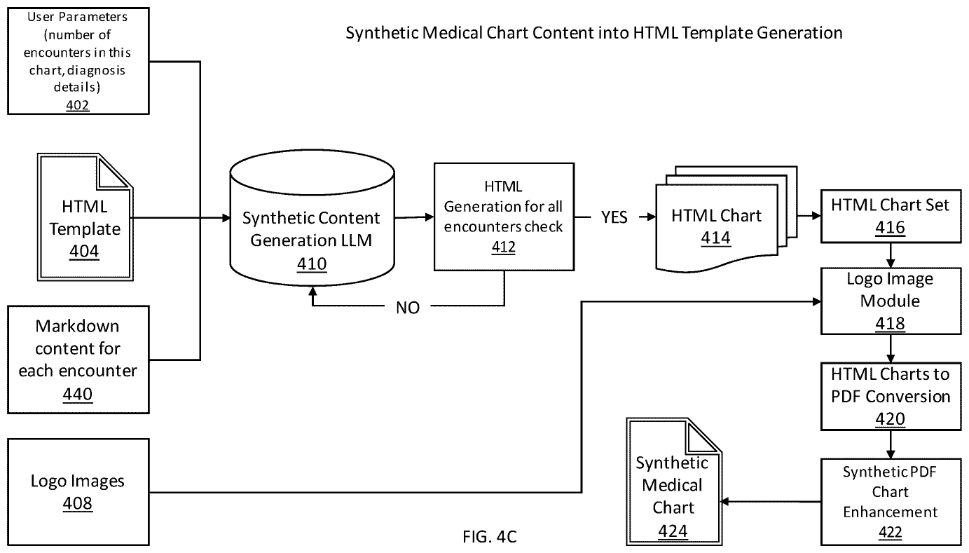

As described in more detail below with reference to FIGS. 4A through 4C, redacted HTML sheet 301 is processed by server 4, which passes the HTML sheet 301 to a LLM along with a prompt. The LLM generates content, including content designed for the fields identified in HTML sheet 301. The generated content is then added to the HTML sheet to create completed synthetic medical record 321. Synthetic medical record includes appropriate content for patient name field 322, date of birth field 324, sex and gender fields 326 and 328, date of service field 330 and visit type field 332. As discussed below, because the LLM was prompted with the field names identified in the HTML sheet, it was given information sufficient to create a synthetic medical chart with content consistent with the named field. E.g., the patient name field 322 is filled in with content that resembles a human name, the date of birth field 324 is filled with a date of birth, in date format and age appropriate for the other content in the cart, the sex field 326 and gender field 328 are filled with appropriate answers to those categories, date of service 330 is filled with a temporally appropriate date formatted record, visit type field 332 is filled with an appropriate categorical response, etc.

Turning now to FIG. 4A, where a process flow diagram of a process of converting a PDF redacted medical record 426 to an HTML template 301, in accordance with one aspect of the invention, is shown. PDF template 426 may be an authentic medical record, with information, such as PHI or PII, redacted from it. However, as also shown in FIG. 3, although the field entries, e.g. those containing PHI or PII, are redacted, the names of the fields would remain unredacted on PDF template 426. The unredacted fields allow the LLM to be instructed on what kinds of content needs to be created.

In some embodiments, PDF template 426 may be converted to PNG images, e.g. by server 4 of FIG. 1, as illustrated in block 428. The PDF pages are converted to PNG images, in such embodiments, to increase the ease with which the LLM may interpret the template.

At block 430, server 4 prompts the LLM to convert the PNG images to HTML code. This HTML code represents an HTML template 301 that will be later used for identifying fields and for formatting the final synthetic health record, as described in more detail below. The HTML template 301 includes fields, or user parameters, where content or information will need to be filled in by subsequent processes or steps in the process as discussed below. User parameters may include, as shown in FIG. 3 above, patient's name 322, date of birth 324 and other identifying features, facility or other provider identifying information, visit information, e.g. date 330 and/or time, medical history information, e.g. symptoms or other conditions or circumstances the patient told the provider or with which the patient presented at the visit, diagnoses, prescriptions, orders and the like. The LLM may be a custom trained LLM designed for performing certain functions of the system described herein, including the conversion from PNG images to HTML code. In other embodiments, the LLM may be a generally available LLM such as ChatGPT. In some embodiments, different LLMs may be used for different system functions described herein, and “the LLM” in the process may also refer to a first LLM for one function and a second LLM for a second function. In other embodiments, the PDF may be converted to HTML code without an intermediate conversion to PNG or an image format.

In some embodiments, the LLM may originate from management server 4 of FIG. 1, while the LLM itself may be accessible at cloud based engine 8, with calls being transmitted from management server 4 to cloud based engine 8 via communications network 22. In some embodiments, user devices 16, 18, or 20 may send requests that the calls be made, and/or other information, to the management server 4 via communication network 22.

Referring back to FIG. 4A, in some embodiments, the system might also utilize an image model 418 to convert text to image to produce logo images 408 to be placed on a final synthetic medical chart. In some embodiments, the text may have been extracted from PDF template 426 prior to conversion. In other embodiments, logo images 408 may be extracted from PDF template 426 and/or PNG images 428 to be reinserted into the final synthetic medical record 321.

Turning now to FIG. 4B, where a flow diagram of a method for singular or multiple encounters between a patient and medical providers, to be placed into a synthetic medical chart, is shown. In some embodiments, the system provides the LLM, at block 410, with the HTML template 301 generated in the previous process as shown in FIG. 4A. The LLM may also be presented with user parameters, which may be included in the template or may be presented apart from the template. User parameters may refer to items of information relating to a synthetic medical record, which may relate to the patient, the facility, the fields needed in the chart or other information the LLM may use to generate relevant content. User parameters may include, but are not limited to, the number of encounters for which charts are needed, necessary patient information and diagnosis details as shown in block 402. The LLM may also be provided with instructions 432, which may include best practice instructions, which may be used to instruct the LLM so that it may more closely mimic how a real health care provider may update a real medical chart.

The LLM is then prompted to produce content for one medical chart for each encounter of the number encounters identified in the user parameters. At block 410, the LLM utilizes the memory from large datasets it is trained on, and the user parameters 402, the fields identified in the HTML template 301, and the instructions 432 to generate synthetic content for the first encounter 436. The information the LLM generates may be associated with the field names in the HTML template 301, such that the LLM generates content that resembles a patient name for a patient name field, a facility name for a facility name field, a medical history narrative for a field that calls for a narrative, etc. Chart content may be created and/or stored in a plain text format to make it more useful for inserting into a synthetic chart in a subsequent step discussed below.

In some embodiments, e.g. when the number of encounters, in the user parameters, is greater than 1, the LLM may need to generate multiple charts representing multiple encounters, e.g., at one or more facilities, by the same patient, e.g., over a fictitious timeline. However, a constraint of the LLM is that it is limited with respect to how many words it can produce. Accordingly, multiple, e.g. iterative, calls to the LLM may be necessary to generate content for the requested number of encounters.

The LLM may need to determine if the system prompted it to generate more than one encounter as identified in the user parameters data represented in block 402, in scenarios where producing all of the data necessary for a comprehensive medical report for the total number of requested encounters cannot be generated in a single output by the LLM. Yet, there is still a need for the comprehensive medical report to be consistent and logically and temporally cohesive for all encounters in the synthetic medical report. In such examples, a chart memory 434 may be created to store the user parameters from the first encounter, and a chart memory summary of the first chart, rather than, or in addition to, storing the full chart content. In some embodiments, the chart memory summary may be created, for storage in the chart memory, via a call to the LLM either simultaneously with, or after, each iteration of content creation. For example, the LLM may be prompted to create both a complete set of chart content for all of the fields of the template, and may also be prompted to create a shorter, e.g. narrative, summary of the chart that the LLM created. The prompt to create the summary may include a maximum length, e.g. in characters, for the summary.

If an LLM were prompted to create a medical chart containing multiple health records for the same fictitious patient, without the use of an iterative process and/or a memory, the patient information would vary in illogical ways from record to record. For example, the same patient may have a different date of birth in different records. Other issue may include a subsequent record failing to address an ailment recorded in a previous record, the medical information in two records may contradict each other, etc. Accordingly, a chart memory, e.g. when used with an iterative process to create each subsequent chart, allows the prompt to be updated to include previous medical and other identifying information about the patient, so that the synthetic charts can be internally consistent temporally, logically, and medically.

The parameters and/or the chart summary may then be input into the LLM at block 410 for logical generation of the succeeding encounters. The chart memory summary is useful to allow the LLM to have a summary of the fictitious patient's health history, from previous charts, so that it can make subsequent chart content that is medically, logically, temporally, etc., consistent between the different encounters. E.g., content may deal with progression over time of the same illness or condition, may reflect that the patient is on the same medications, may reflect recovery in a subsequent chart from a condition identified in a prior chart, etc. The chart summary, in the chart memory, allows the LLM to create such consistent charts without having to pass the entire corpus of prior charts to the LLM when creating the next iterative chart.

The system may prompt the LLM to generate and/or update the chart memory summary after every encounter. The process of updating the chart memory summary may include summarizing the patient's diagnosis detail from the chart content and storing it into the chart memory 434. The chart memory summary along with the user parameters stored at block 434 may be input into the LLM, e.g., in another iteration, at block 410. The LLM may then generate the synthetic content, e.g., in a text format, for each of the succeeding encounters by a process of creating the chart memory summary from the previous encounter and inputting it to the LLM to produce content for the next encounter. When the LLM has created content for the number of encounters that was requested, the encounter summaries are output, as depicted by block 438. The synthetic content for the first and following encounters generated, e.g. in a text format, may then be combined all together as shown at block 440. In other embodiments, content for each encounter may be stored separately from other encounters.

Turning now to FIG. 4C, where a flow diagram illustrating the merging of the synthetic content generated FIG. 4A with the HTML template 301 generated in FIG. 4B, is shown. In some embodiments, the system may provide the LLM with the HTML template 301, user parameters 402 that may include, but are not limited to the number of encounters, necessary patient information and diagnosis details as shown by block 402 and the text content 440 generated for each encounter as depicted in FIG. 4B. The LLM at block 410 utilizes all the information given by the system and combines the first encounter provided by block 440 to the HTML template 301, e.g. by inserting the content into the section of the template where content of that type, e.g. patient name, medical narrative, should visually reside in the template.

The system then checks at block 412 if all the text representing the encounters have been merged into the HTML template 301. If not, the system prompts the LLM 410 to combine the next encounter to the HTML template. The system repeats this process until the text content of each encounter is combined to the HTML template 301. If all the text content is combined to the HTML templates, the system may merge all the HTML templates generated at block 412 together to form a HTML chart as shown by block 414. In some embodiments, the system might process the HTML charts to have consistent CSS styling at block 416 to make the chart clear and realistic. The system may also insert one or more logo images 408 to the HTML chart as depicted by the block 418. The system may convert the HTML chart to a single long PDF chart as depicted by the process in block 420.

In some examples, the system may enhance the PDF chart to make it look realistic by adding components including, but not limited to image noise and other artifacts at block 422.

The system then outputs the final PDF chart—a synthetic medical chart 321—containing synthetic medical content for a single encounter or multiple encounters. The synthetic chart is formatted to appear similar to authentic charts, and may include elements of real charts such as real logos. The synthetic chart may also include synthetic artifacts, e.g. to simulate imperfect scanning of a paper chart and other difficulties that software applications, being tested on the synthetic charts, may encounter with real charts.

It will be appreciated that creation of synthetic medical charts as disclosed herein, particularly on large datasets intended to be used with the disclosed embodiments/to generate trained models used in the disclosed embodiments, is only possible with the aid of computer-assisted machine-learning algorithms and techniques, such as LLMs. In some embodiments, machine learning processes including LLMs are used to perform operations that cannot practically be performed by a human, either mentally or with assistance, such as generating synthetic content based on a template and a previous medical history stored as a summary in a memory. It will be appreciated that a variety of machine learning techniques can be used alone or in combination to generate synthetic medical charts that are voluminous enough to test software designed to analyze real medical charts having a similar volume of content.

In some embodiments, the LLM may be prompted with a different prompt for template generation, content generation, and inserting content into templates. Template generation prompts may include a document, along with an instruction to make an HTML webpage to reproduce the layout and format of the document. Template generation prompts may include instructions to include and ensure the quality and matching of CSS styles, alignments, tables, font sizes, and colors, to increase the realism of the template.

Content generation prompts may include prompts may include prompts to generate chart content, and prompts to generate or update the chart memory, for use in subsequent prompts to generate subsequent chart content. Content generation prompts may include instructions to Generate fictitious text content for an included HTML Template of a medical chart with progress note outpatient summaries. Content generation prompts may include instructions to adhere to the HTML Template provided but write the answer in text, e.g. plain text. Instructions may also be included to use the details (names, contact info) provided in the Details section in the prompt, unless it is blank, and then the LLM may be instructed to make up its own details. Content generation prompts may also include instruction to ensure that the fictitious visit follows logically from the Summary of Past Visits section included in the prompt, unless that section is blank, then consider this the first visit with this particular patient. Content generation prompts may also include instructions to include where/if generated parts of the content fits into the Template, e.g. current medications, past medical history, family history, social history, review of systems, chief complaint, history of present illness, vital signs, physical exam, assessment, treatments/plan, follow up, etc. Instructions relating to the verbosity of the content may also be included.

Chart memory prompts may include the content generated by a content generation prompt, and/or a previous chart memory summary generated by the LLM in response to a previous chart memory prompt, along with instructions to summarize the medical visit in included medical chart, e.g. in a specified shortened format, e.g., fewer than 5 sentences. Chart memory prompts may include instructions to include the new information from this visit, but omit the history of past visits. An instruction to include the date of the summarized visit in the summary may also be included.

Prompts for placing content in templates may include a template, which may have some fields filled in, e.g. from prior iterations of the process on synthetic medical charts on the same fictitious patient. Prompts for placing content in templates may also include instructions for the LLM that an include HTML web page has outdated content, and instructing the LLM to replace the content of the page with generated content, without changing the design, styling, or formatting of the page, keeping the CSS exactly the same, and filling in placeholders with details (name, DOB) in any header or footer elements appropriately.

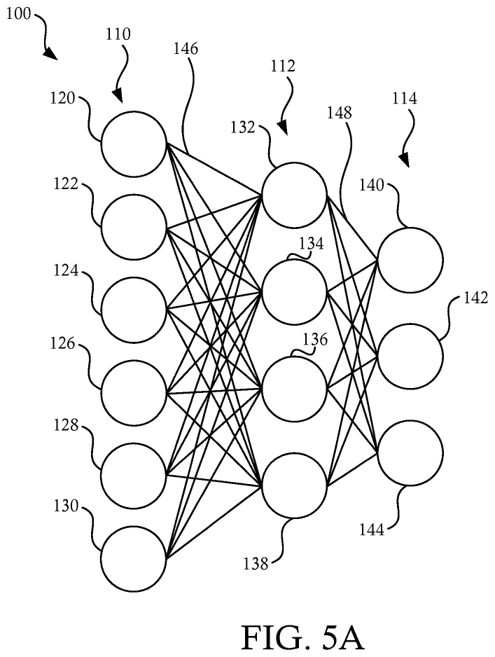

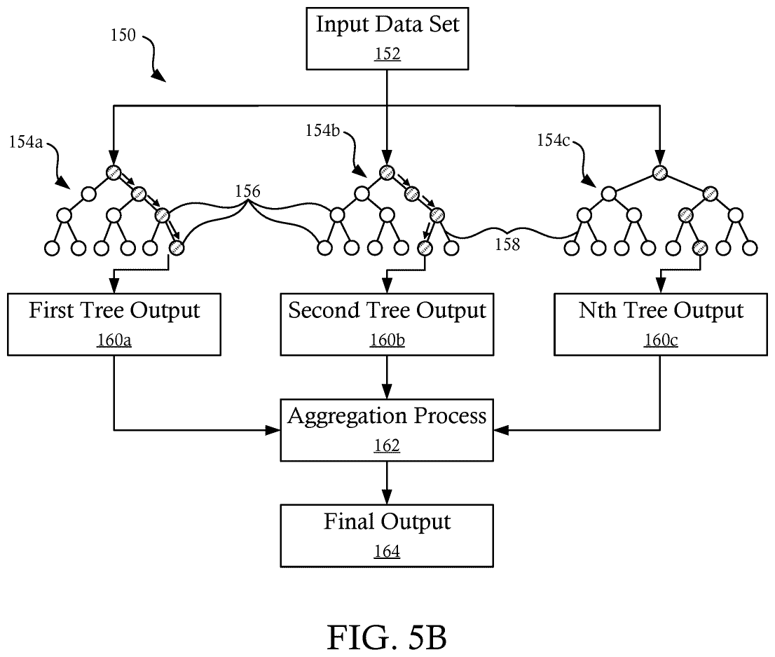

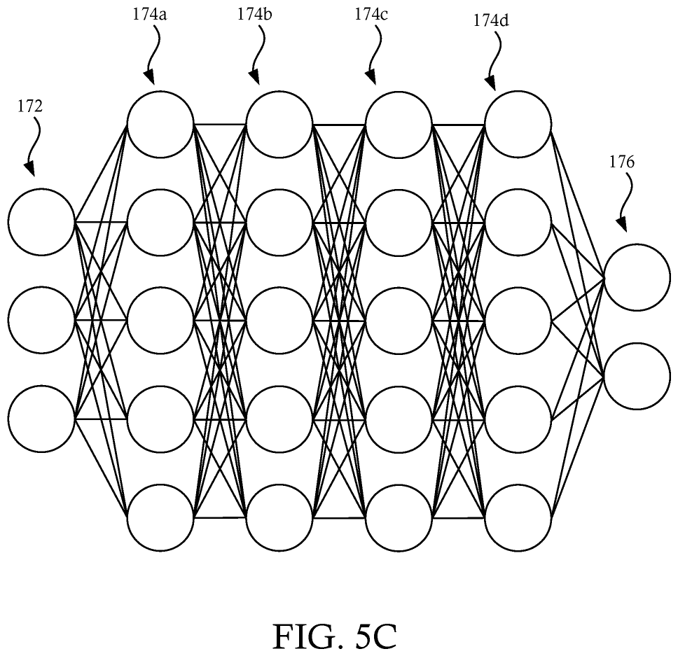

FIG. 5A illustrates an artificial neural network 100, in accordance with some embodiments. Alternative terms for “artificial neural network” are “neural network,” “artificial neural net,” “neural net,” or “trained function.” The neural network 100 comprises nodes 120-144 and edges 146-148, wherein each edge 146-148 is a directed connection from a first node 120-138 to a second node 132-144. In general, the first node 120-138 and the second node 132-144 are different nodes, although it is also possible that the first node 120-138 and the second node 132-144 are identical. For example, in FIG. 5A the edge 146 is a directed connection from the node 120 to the node 132, and the edge 148 is a directed connection from the node 132 to the node 140. An edge 146-148 from a first node 120-138 to a second node 132-144 is also denoted as “ingoing edge” for the second node 132-144 and as “outgoing edge” for the first node 120-138.

The nodes 120-144 of the neural network 100 may be arranged in layers 110-114, wherein the layers may comprise an intrinsic order introduced by the edges 146-148 between the nodes 120-144 such that edges 146-148 exist only between neighboring layers of nodes. In the illustrated embodiment, there is an input layer 110 comprising only nodes 120-130 without an incoming edge, an output layer 114 comprising only nodes 140-144 without outgoing edges, and a hidden layer 112 in-between the input layer 110 and the output layer 114. In general, the number of hidden layer 112 may be chosen arbitrarily and/or through training. The number of nodes 120-130 within the input layer 110 usually relates to the number of input values of the neural network, and the number of nodes 140-144 within the output layer 114 usually relates to the number of output values of the neural network.

In particular, a (real) number may be assigned as a value to every node 120-144 of the neural network 100. Here,

$${x}_{i}^{( n )}$$

denotes the value of the i-th node 120-144 of the n-th layer 110-114. The values of the nodes 120-130 of the input layer 110 are equivalent to the input values of the neural network 100, the values of the nodes 140-144 of the output layer 114 are equivalent to the output value of the neural network 100. Furthermore, each edge 146-148 may comprise a weight being a real number, in particular, the weight is a real number within the interval [−1, 1], within the interval [0, 1], and/or within any other suitable interval. Here,

$${w}_{i , j}^{( m , n )}$$

denotes the weight of the edge between the i-th node 120-138 of the m-th layer 110, 112 and the j-th node 132-144 of the n-th layer 112, 114. Furthermore, the abbreviation

$${w}_{i , j}^{( n )}$$

is defined for the weight

$${w}_{i , j}^{( n , n + 1 )} .$$

In particular, to calculate the output values of the neural network 100, the input values are propagated through the neural network. In particular, the values of the nodes 132-144 of the (n+1)-th layer 112, 114 may be calculated based on the values of the nodes 120-138 of the n-th layer 110, 112 by

$${x}_{j}^{( n + 1 )} = f ( {\sum }_{i} {x}_{i}^{( n )} \cdot {w}_{i , j}^{( n )} )$$

Herein, the function f is a transfer function (another term is “activation function”). Known transfer functions are step functions, sigmoid function (e.g., the logistic function, the generalized logistic function, the hyperbolic tangent, the Arctangent function, the error function, the smooth step function) or rectifier functions. The transfer function is mainly used for normalization purposes.

In particular, the values are propagated layer-wise through the neural network, wherein values of the input layer 110 are given by the input of the neural network 100, wherein values of the hidden layer(s) 112 may be calculated based on the values of the input layer 110 of the neural network and/or based on the values of a prior hidden layer, etc.

In order to set the values

$${w}_{i , j}^{( m , n )}$$

for the edges, the neural network 100 has to be trained using training data. In particular, training data comprises training input data and training output data. For a training step, the neural network 100 is applied to the training input data to generate calculated output data. In particular, the training data and the calculated output data comprise a number of values, said number being equal with the number of nodes of the output layer.

In particular, a comparison between the calculated output data and the training data is used to recursively adapt the weights within the neural network 100 (backpropagation algorithm). In particular, the weights are changed according to

$${w}_{i , j}^{′ ( n )} = {w}_{i , j}^{( n )} - γ \cdot {δ}_{j}^{( n )} \cdot {x}_{i}^{( n )}$$

wherein γ is a learning rate, and the numbers

$${δ}_{j}^{( n )}$$

may be recursively calculated as

$${δ}_{j}^{( n )} = ( {\sum }_{k} {δ}_{k}^{( n + 1 )} \cdot {w}_{j , k}^{( n + 1 )} ) \cdot {f}^{′} ( {\sum }_{i} {x}_{i}^{( n )} \cdot {w}_{i , j}^{( n )} )$$

based on

$${δ}_{j}^{( n + 1 )} ,$$

if the (n+1)-th layer is not the output layer, and

$${δ}_{j}^{( n )} = ( {x}_{k}^{( n + 1 )} - {t}_{j}^{( n + 1 )} ) \cdot {f}^{′} ( {\sum }_{i} {x}_{i}^{( n )} \cdot {w}_{i , j}^{( n )} )$$

if the (n+1)-th layer is the output layer 114, wherein f′ is the first derivative of the activation function, and

$${y}_{j}^{( n + 1 )}$$

is the comparison training value for the j-th node of the output layer 114.

FIG. 5B illustrates a tree-based neural network 150, in accordance with some embodiments. In particular, the tree-based neural network 150 is a random forest neural network, though it will be appreciated that the discussion herein is applicable to other decision tree neural networks. The tree-based neural network 150 includes a plurality of trained decision trees 154a-154c each including a set of nodes 156 (also referred to as “leaves”) and a set of edges 158 (also referred to as “branches”).

Each of the trained decision trees 154a-154c may include a classification and/or a regression tree (CART). Classification trees include a tree model in which a target variable may take a discrete set of values, e.g., may be classified as one of a set of values. In classification trees, each leaf 156 represents class labels and each of the branches 158 represents conjunctions of features that connect the class labels. Regression trees include a tree model in which the target variable may take continuous values (e.g., a real number value).

In operation, an input data set 152 including one or more features or attributes is received. A subset of the input data set 152 is provided to each of the trained decision trees 154a-154c. The subset may include a portion of and/or all of the features or attributes included in the input data set 152. Each of the trained decision trees 154a-154c is trained to receive the subset of the input data set 152 and generate a tree output value 160a-160c, such as a classification or regression output. The individual tree output value 160a-160c is determined by traversing the trained decision trees 154a-154c to arrive at a final leaf (or node) 156.

In some embodiments, the tree-based neural network 150 applies an aggregation process 162 to combine the output of each of the trained decision trees 154a-154c into a final output 164. For example, in embodiments including classification trees, the tree-based neural network 150 may apply a majority-voting process to identify a classification selected by the majority of the trained decision trees 154a-154c. As another example, in embodiments including regression trees, the tree-based neural network 150 may apply an average, mean, and/or other mathematical process to generate a composite output of the trained decision trees. The final output 164 is provided as an output of the tree-based neural network 150.

FIG. 5C illustrates a deep neural network (DNN) 170, in accordance with some embodiments. The DNN 170 is an artificial neural network, such as the neural network 100 illustrated in conjunction with FIG. 3, that includes representation learning. The DNN 170 may include an unbounded number of (e.g., two or more) intermediate layers 174a-174d each of a bounded size (e.g., having a predetermined number of nodes), providing for practical application and optimized implementation of a universal classifier. Each of the layers 174a-174d may be heterogenous. The DNN 170 may be configured to model complex, non-linear relationships. Intermediate layers, such as intermediate layer 174c, may provide compositions of features from lower layers, such as layers 174a, 174b, providing for modeling of complex data.

In some embodiments, the DNN 170 may be considered a stacked neural network including multiple layers each configured to execute one or more computations. The computation for a network with L hidden layers may be denoted as:

$$f ( x ) = f [ {a}^{( L + 1 )} ( {h}^{( L )} ( {a}^{( L )} ( … ( {h}^{( 2 )} ( {a}^{( 2 )} ( {h}^{( 1 )} ( {a}^{( 1 )} ( x ) ) ) ) ) ) ) ) ]$$

where a^{(l)}(x) is a preactivation function and h^{(l)}(x) is a hidden-layer activation function providing the output of each hidden layer. The preactivation function a^{(l)}(x) may include a linear operation with matrix W^{(l)} and bias b^{(l)} where:

$${a}^{( l )} ( x ) = {W}^{( l )} x + {b}^{( l )}$$

In some embodiments, the DNN 170 is a feedforward network in which data flows from an input layer 172 to an output layer 176 without looping back through any layers. In some embodiments, the DNN 170 may include a backpropagation network in which the output of at least one hidden layer is provided, e.g., propagated, to a prior hidden layer. The DNN 170 may include any suitable neural network, such as a self-organizing neural network, a recurrent neural network, a convolutional neural network, a modular neural network, and/or any other suitable neural network.

In some embodiments, a DNN 170 may include a neural additive model (NAM). An NAM includes a linear combination of networks, each of which attends to (e.g., provides a calculation regarding) a single input feature. For example, a NAM may be represented as:

$$y = β + {f}_{1} ( {x}_{1} ) + {f}_{2} ( {x}_{2} ) + ⋯ + {f}_{K} ( {x}_{K} )$$

where β is an offset and each fᵢ is parametrized by a neural network. In some embodiments, the DNN 170 may include a neural multiplicative model (NMM), including a multiplicative form for the NAM mode using a log transformation of the dependent variable y and the independent variable x:

$$y = {e}^{β} {e}^{f ( logx )} {e}^{{\sum }_{i} {f}_{i}^{d} ( {d}_{i} )}$$

where d represents one or more features of the independent variable x.

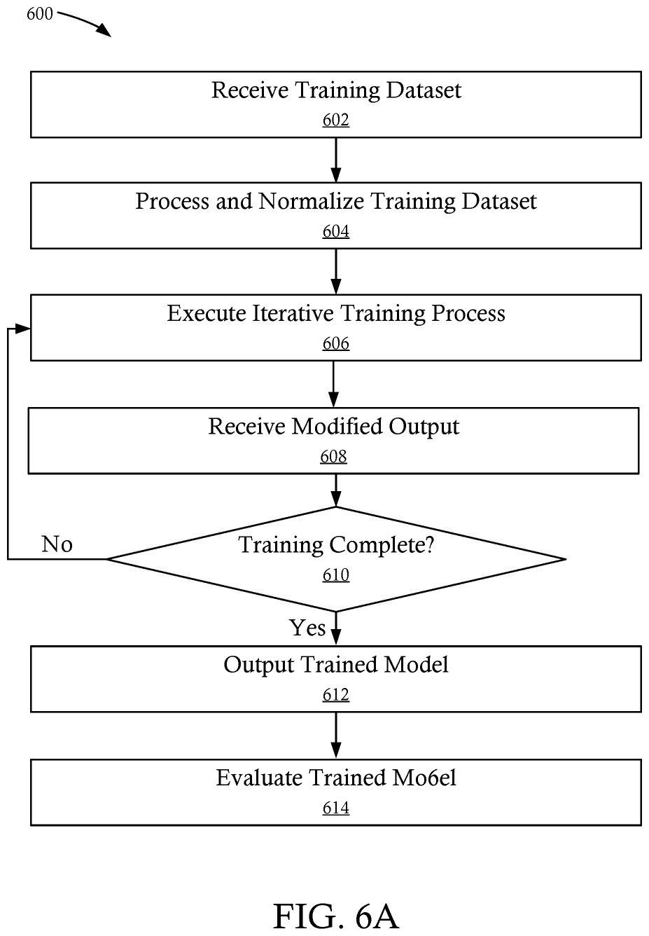

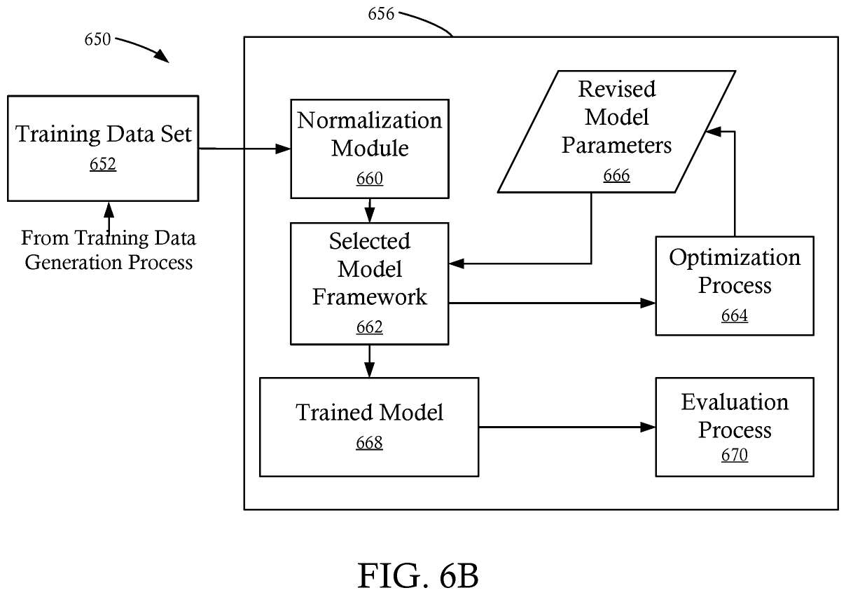

In some embodiments, an LLM can include and/or implement one or more trained models. In some embodiments, one or more trained models can be generated using an iterative training process based on a training dataset. FIG. 6A illustrates a method 600 for generating a trained model, such as a trained LLM model for content generation, template generation, or merging content into a template, in accordance with some embodiments. FIG. 6B is a process flow 650 illustrating various steps of the method 600 of generating a trained model, in accordance with some embodiments. At step 602, a training dataset 652 is received by a system, such as a processing device 10. The training dataset 652 can include labeled and/or unlabeled data. For example, in some embodiments, user parameters and previously generated synthetic medical charts may be provided for use in training a model.

At optional step 604, the received training dataset 652 is processed and/or normalized by a normalization module 660. For example, in some embodiments, the training dataset 652 can be augmented by imputing or estimating missing values of one or more features associated with user parameters. In some embodiments, processing of the received training dataset 652 includes outlier detection configured to remove data likely to skew training. In some embodiments, processing of the received training dataset 652 includes removing features that have limited value with respect to training of the LLM.

At step 606, an iterative training process is executed to train a selected model framework 662. The selected model framework 662 can include an untrained (e.g., base) machine learning model, such as an LLM and/or a partially or previously trained model (e.g., a prior version of a trained model).

The training process is an iterative process that generates set of revised model parameters 666 during each iteration. The set of revised model parameters 666 can be generated by applying an optimization process 664 to the cost function of the selected model framework 662. The optimization process 664 can be configured to reduce the cost value (e.g., reduce the output of the cost function) at each step by adjusting one or more parameters during each iteration of the training process.

After each iteration of the training process, at step 608, a determination is made whether the training process is complete. The determination at step 608 can be based on any suitable parameters. For example, in some embodiments, a training process can complete after a predetermined number of iterations. As another example, in some embodiments, a training process can complete when it is determined that the cost function of the selected model framework 662 has reached a minimum, such as a local minimum and/or a global minimum.

At step 610, a trained model 668, such as a trained LLM, is output and provided for use in a method of generating templates, content, or charts, such as the methods discussed above with respect to FIGS. 4A-4C. At optional step 612, a trained model 668 can be evaluated by an evaluation process 670. A trained model can be evaluated based on any suitable metrics, such as, for example, an F or F1 score, normalized discounted cumulative gain (NDCG) of the model, mean reciprocal rank (MRR), mean average precision (MAP) score of the model, and/or any other suitable evaluation metrics. Although specific embodiments are discussed herein, it will be appreciated that any suitable set of evaluation metrics can be used to evaluate a trained model.

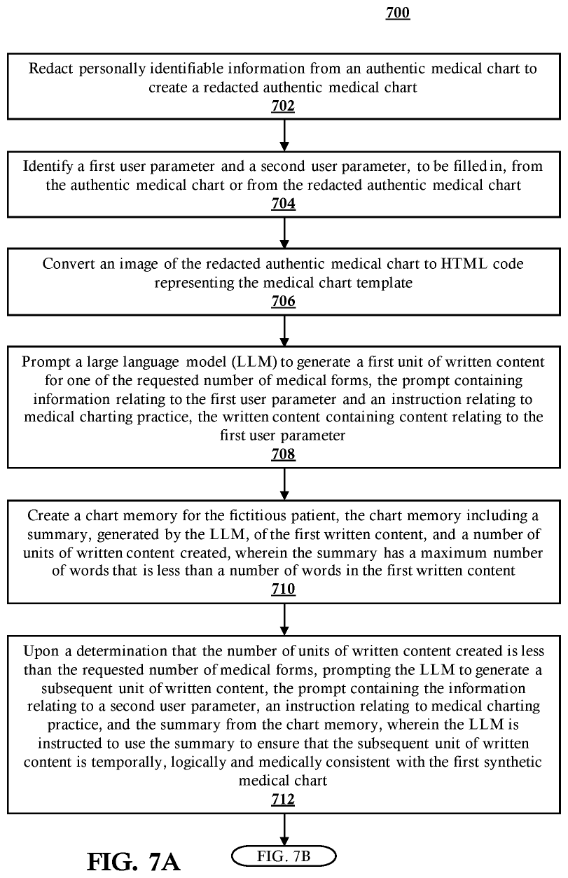

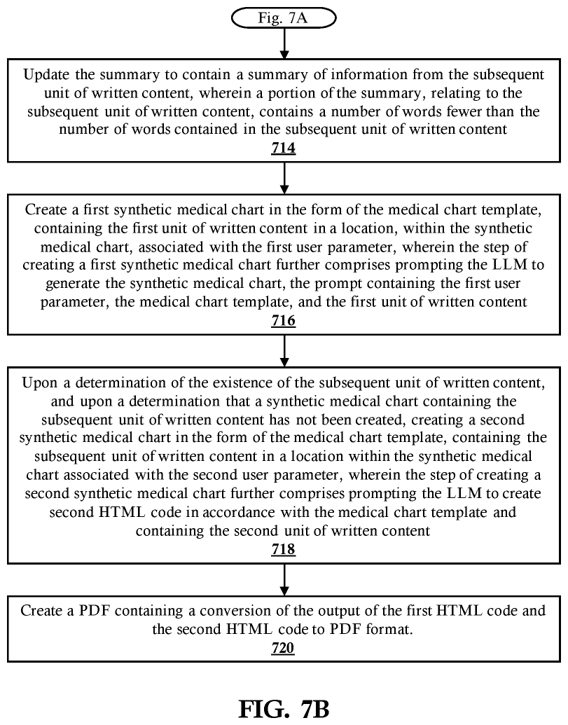

Turning now to FIGS. 7A and 7B, wherein a flow diagram of a method for creating a requested number of medical record forms relating to a single fictitious patient, each of the medical record forms representing a fictitious encounter between the fictitious patent and a medical provider, in accordance with one aspect of the present disclosure is shown.

The method includes redacting (702) personally identifiable information from an authentic medical chart to create a redacted authentic medical chart. The method further includes identifying (704) a first user parameter and a second user parameter, to be filled in, from the authentic medical chart or from the redacted authentic medical chart, and converting (706) an image of the redacted authentic medical chart to HTML code representing the medical chart template. The method further includes generating synthetic medical content, the generating the synthetic medical content further comprising prompting (708) a large language model (“LLM”) to generate a first unit of written content for one of the requested number of medical forms, the prompt containing information relating to the first user parameter and an instruction relating to medical charting practice, the written content containing content relating to the first user parameter, and creating (710) a chart memory for the fictitious patient, the chart memory including a summary, generated by the LLM, of the first written content, and a number of units of written content created, wherein the summary has a maximum number of words that is less than a number of words in the first written content.

The content generation further includes, upon a determination that the number of units of written content created is less than the requested number of medical forms, prompting (712) the LLM to generate a subsequent unit of written content, the prompt containing the information relating to a second user parameter, an instruction relating to medical charting practice, and the summary from the chart memory, wherein the LLM is instructed to use the summary to ensure that the subsequent unit of written content is temporally, logically and medically consistent with the first synthetic medical chart, and updating (714) the summary to contain a summary of information from the subsequent unit of written content, wherein a portion of the summary, relating to the subsequent unit of written content, contains a number of words fewer than the number of words contained in the subsequent unit of written content.

The method further includes creating (716) a first synthetic medical chart in the form of the medical chart template, containing the first unit of written content in a location, within the synthetic medical chart, associated with the first user parameter, wherein the step of creating a first synthetic medical chart further includes prompting the LLM to generate the synthetic medical chart, the prompt containing the first user parameter, the medical chart template, and the first unit of written content. Upon a determination of the existence of the subsequent unit of written content, and upon a determination that a synthetic medical chart containing the subsequent unit of written content has not been created, the method includes creating (718) a second synthetic medical chart in the form of the medical chart template, containing the subsequent unit of written content in a location within the synthetic medical chart associated with the second user parameter, wherein the step of creating a second synthetic medical chart further comprises prompting the LLM to create second HTML code in accordance with the medical chart template and containing the second unit of written content. Finally, the method includes creating (720) a PDF containing a conversion of the output of the first HTML code and the second HTML code to PDF format.

Although the subject matter has been described in terms of exemplary embodiments, it is not limited thereto. Rather, the appended claims should be construed broadly, to include other variants and embodiments, which may be made by those skilled in the art.

What is claimed is:

1. A computer-implemented method for creating a requested number of medical record forms relating to a single fictitious patient, each of the medical record forms representing a fictitious encounter between the fictitious patent and a medical provider, the method comprising:
creating a medical chart template, the creating a medical chart template further comprising:
redacting personally identifiable information from an authentic medical chart to create a redacted authentic medical chart;
identifying a first user parameter and a second user parameter, to be filled in, from the authentic medical chart or from the redacted authentic medical chart; and
converting an image of the redacted authentic medical chart to HTML code representing the medical chart template;
generating synthetic medical content, the generating the synthetic medical content further comprising;
prompting a large language model (“LLM”) to generate a first unit of written content for one of the requested number of medical forms, the prompt containing information relating to the first user parameter and an instruction relating to medical charting practice, the written content containing content relating to the first user parameter;
creating a chart memory for the fictitious patient, the chart memory including a summary, generated by the LLM, of the first written content, and a number of units of written content created, wherein the summary has a maximum number of words that is less than a number of words in the first written content;
upon a determination that the number of units of written content created is less than the requested number of medical forms, prompting the LLM to generate a subsequent unit of written content, the prompt containing the information relating to a second user parameter, an instruction relating to medical charting practice, and the summary from the chart memory, wherein the LLM is instructed to use the summary to ensure that the subsequent unit of written content is temporally, logically and medically consistent with the first unit of written content;
updating the summary to contain a summary of information from the subsequent unit of written content.

2. The method of claim 1, further comprising the step of creating a first synthetic medical chart in the form of the medical chart template, containing the first unit of written content in a location, within the synthetic medical chart, associated with the first user parameter.

3. The method of claim 2, wherein the step of creating a synthetic medical chart further comprises prompting the LLM to generate the synthetic medical chart, the prompt containing the first user parameter, the medical chart template, and the first unit of written content.

4. The method of claim 3, further comprising the step of, upon a determination of the existence of the subsequent unit of written content, and upon a determination that a synthetic medical chart containing the subsequent unit of written content has not been created, creating a second synthetic medical chart in the form of the medical chart template, containing the subsequent unit of written content in a location within the synthetic medical chart associated with the second user parameter.

5. The method of claim 4, wherein the step of creating a first synthetic medical chart further comprises prompting the LLM to create first HTML code in accordance with the medical chart template and containing the first unit of written content.

6. The method of claim 5, wherein the step of creating a second synthetic medical chart further comprises prompting the LLM to create second HTML code in accordance with the medical chart template and containing the subsequent unit of written content.

7. The method of claim 6, further comprising creating a PDF containing a conversion of the output of the first HTML code and the second HTML code to PDF format.

8. A system for creating a requested number of medical record forms relating to a single fictitious patient, each of the medical record forms representing a fictitious encounter between the fictitious patent and a medical provider, comprising:
a non-transitory memory;
a processor communicatively coupled to the non-transitory memory, wherein the processor is configured to read a set of instructions to:
create a medical chart template, the creating a medical chart template further comprising:
redacting personally identifiable information from an authentic medical chart to create a redacted authentic medical chart;
identifying a first user parameter and a second user parameter, to be filled in, from the authentic medical chart or from the redacted authentic medical chart; and
converting an image of the redacted authentic medical chart to HTML code representing the medical chart template;
generate synthetic medical content, the generating the synthetic medical content further comprising;
prompting a large language model (“LLM”) to generate a first unit of written content for one of the requested number of medical forms, the prompt containing information relating to the first user parameter and an instruction relating to medical charting practice, the written content containing content relating to the first user parameter;
creating a chart memory for the fictitious patient, the chart memory including a summary, generated by the LLM, of the first written content, and a number of units of written content created, wherein the summary has a maximum number of words that is less than a number of words in the first written content;
upon a determination that the number of units of written content created is less than the requested number of medical forms, prompting the LLM to generate a subsequent unit of written content, the prompt containing the information relating to a second user parameter, an instruction relating to medical charting practice, and the summary from the chart memory, wherein the LLM is instructed to use the summary to ensure that the subsequent unit of written content is temporally, logically and medically consistent with the first unit of written content;
updating the summary to contain a summary of information from the subsequent unit of written content.

9. The system of claim 8, the instructions further comprising an instruction to create a first synthetic medical chart in the form of the medical chart template, containing the first unit of written content in a location, within the synthetic medical chart, associated with the first user parameter.

10. The system of claim 9, wherein the instruction to create a synthetic medical chart further comprises prompting the LLM to generate the synthetic medical chart, the prompt containing the first user parameter, the medical chart template, and the first unit of written content.

11. The system of claim 10, the instructions further comprising an instruction to, upon a determination of the existence of the subsequent unit of written content, and upon a determination that a synthetic medical chart containing the subsequent unit of written content has not been created, create a second synthetic medical chart in the form of the medical chart template, containing the subsequent unit of written content in a location within the synthetic medical chart associated with the second user parameter.

12. The system of claim 11, wherein the step of creating a first synthetic medical chart further comprises prompting the LLM to create first HTML code in accordance with the medical chart template and containing the first unit of written content.

13. The system of claim 12, wherein the step of creating a second synthetic medical chart further comprises prompting the LLM to create second HTML code in accordance with the medical chart template and containing the subsequent unit of written content.

14. The system of claim 13, further comprising creating a PDF containing a conversion of the output of the first HTML code and the second HTML code to PDF format.

15. A non-transitory computer readable medium having instructions stored thereon, wherein the instructions, when executed by at least one processor, cause at least one device to perform operations comprising:
creating a medical chart template, the creating a medical chart template further comprising:
redacting personally identifiable information from an authentic medical chart to create a redacted authentic medical chart;
identifying a first user parameter and a second user parameter, to be filled in, from the authentic medical chart or from the redacted authentic medical chart; and
converting an image of the redacted authentic medical chart to HTML code representing the medical chart template;
generating synthetic medical content, the generating the synthetic medical content further comprising;
prompting a large language model (“LLM”) to generate a first unit of written content for one of the requested number of medical forms, the prompt containing information relating to the first user parameter and an instruction relating to medical charting practice, the written content containing content relating to the first user parameter;
creating a chart memory for the fictitious patient, the chart memory including a summary, generated by the LLM, of the first written content, and a number of units of written content created, wherein the summary has a maximum number of words that is less than a number of words in the first written content;
upon a determination that the number of units of written content created is less than the requested number of medical forms, prompting the LLM to generate a subsequent unit of written content, the prompt containing the information relating to a second user parameter, an instruction relating to medical charting practice, and the summary from the chart memory, wherein the LLM is instructed to use the summary to ensure that the subsequent unit of written content is temporally, logically and medically consistent with the first unit of written content;
updating the summary to contain a summary of information from the subsequent unit of written content.

16. The medium of claim 15, the operations further comprising creating a first synthetic medical chart in the form of the medical chart template, containing the first unit of written content in a location, within the synthetic medical chart, associated with the first user parameter.

17. The medium of claim 16, wherein the operation of creating a synthetic medical chart further comprises prompting the LLM to generate the synthetic medical chart, the prompt containing the first user parameter, the medical chart template, and the first unit of written content.

18. The medium of claim 17, the operations further comprising, upon a determination of the existence of the subsequent unit of written content, and upon a determination that a synthetic medical chart containing the subsequent unit of written content has not been created, creating a second synthetic medical chart in the form of the medical chart template, containing the subsequent unit of written content in a location within the synthetic medical chart associated with the second user parameter.

19. The medium of claim 18, wherein the operation of creating a first synthetic medical chart further comprises prompting the LLM to create first HTML code in accordance with the medical chart template and containing the first unit of written content.

20. The medium of claim 19, wherein the operation of creating a second synthetic medical chart further comprises prompting the LLM to create second HTML code in accordance with the medical chart template and containing the subsequent unit of written content.
